(19) United States
(12) Patent Application Publication
(10) Pub. No.: US 2002/0023642 A1
(43) Pub. Date: Feb. 28, 2002

(54) DEVICE FOR ANAESTHETIC SYSTEMS

(76) Inventors: DAN HOLMSTEN, KALLERED (SE); MONICA ALLARD, SODERFORS (SE)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 09/043,062

(22) PCT Filed: Sep. 12, 1996
(86) PCT No.: PCT/SE96/01139

(30) Foreign Application Priority Data
Sep. 12, 1995 (SE) 9503128-2
Oct. 2, 1995 (SE) 9503397-3

(51) Int. Cl. A61M 15/00; A61M 16/10; A62B 7/10; A62B 23/02

(52) U.S. Cl. 128/203.12

(57) ABSTRACT

Method and device for handling a carbon dioxideabsorbing compound in anaesthetic systems of the circle system type. The anaesthetic system is arranged with an openable container (10) arranged for gas passage in such a way that air mixed with carbon dioxide from the patient who is to be connected to the system, passes through the container in order to be returned to the patient. According to the invention, a quantity of carbon dioxide-absorbing compound (16) is provided with a gas permeable cover (17). The unit, intended for carbon dioxide absorption, designed in this way is placed inside the container (10). When it is considered to be consumed, it is removed from the container and replaced with a new one.

### TECHNICAL FIELD

The invention relates to a device for anaesthetic systems. The invention is utilized in breathing systems for anaesthesia of the circle type, wherein the carbon dioxide exhaled by the patient is eliminated by means of a lime compound, while other anaesthetic gases and oxygen are reused by the patient (re-breathing system with carbon dioxide absorption). Circle systems provide very good heating and moistening of the inhaled gas.

The present invention relates to pulmonary ventilation for patients under anaesthesia, with the use of breathing systems associated with anaesthetic apparatuses and more precisely to a so called circle system, in which a carbon dioxide absorber is included in order to eliminate the carbon dioxide content in the inhalation air. Particularly, the invention relates to an absorber container which by means of one single connection, is connected to the breathing system.

Thus, the invention does not relate to the so called Mapleson systems in which exhaled carbon dioxide is flushed away by means of high fresh gas flows of oxygen and anaesthetic gas (system for partial re-breathing without carbon dioxide absorption). Nor has the invention any use in systems with no re-breathing, wherein each breath only contains previously unused gas.

When a patient is under anaesthetic, it is of a fundamental importance that the respiration and the ventilation of the patient can be controlled completely. Above all, the oxygen supply to the blood and the carbon dioxide evacuation from the alveoli of the lungs must function satisfactorily. Furthermore, it must be possible to add drugs or other gaseous additives to the inhalation air. Therefore, the ventilation of a patient must be possible to execute while completing the above mentioned tasks, and also while keeping the lung sufficiently expanded in order to avoid that it collapses.

### STATE OF THE ART

Presently, the most common system is the circle system in which the fresh gas may be adapted to the needs of the patient, so called low-flow anaesthesia, which implies large gas savings in comparison to the other breathing systems. The basic principle is that exhaled gas is purified from carbon dioxide in a chemical way by means of a lime absorber or a molecular sieve, while generating moisture and heat which provides the inhaled gas with a good conditioning. The other gases, oxygen and nitrous oxide and/or other anaesthetic gases, are reused by the patient. The absorber compound is provided with an indicator dye which changes when the ability of the compound to absorb carbon dioxide has ceased. As a rule, the dye changes from white to violet or from pink to white when the carbon dioxide-absorbing ability of the compound has ceased. In order to make it possible to observe the change in colour during clinical use, the compound is placed in a transparent container. The device for carbon dioxide absorption is designed technically according to two main principles:

Multiple-use containers, as a rule containing between 1 and 3 kg of absorber compound which, after consumption, is discarded whereafter the container is refilled with new absorber compound in bulk form.

Disposable containers, which are pre-filled in the factory, as a rule containing about 0.5-1 kg absorber compound. After consumption, the container with its contents is discarded and replaced by a new one.

The multiple-use container is characterized in low costs for the user (the hospital) while the handling of used and fresh absorber compound is associated with environmental problems, primarily in the form of the spread of absorber dust in the working environment. The absorber dust, which is strongly alkaline, has a toxic effect on the respiratory system of the staff. Technically, the bulk filling is also associated with problems to obtain the correct quantity and packing degree in the container. Change of absorber compound during an anaesthesia is not possible since the filling requires considerable time during which, in that case, the anaesthesia has to be interrupted. The large quantity of absorber compound requires large containers which implies impenetrability problems in the circle system and slow heating and moistening performance of the gas which is to be inhaled by the patient.

The disposable container is characterized in higher costs for the user (the hospital) while the handling is extremely hygienic since the entire container with its contents is replaced when the compound has been consumed. This implies an elimination of the dust problems and change of absorber compound/container may be performed with anaesthesia in progress. The heating and moistening quaities are excellent because of the small container with the small quantity of absorber compound. The leakage problems are very small.

The consumed absorber compound poses no environmental problem but may practically be regarded as a soil improvement agent (lime), while the disposable container, made of plastic, of course constitutes a larger problem from a recycling point of view.

The anaesthetic gases which are added to the inhalation air are often expensive and a high consumption of such gases implies both practical problems and large costs. By using an essentially closed system in which the exhaled air after purification from carbon dioxide gas and an addition of a small amount of anaesthetic gas is returned to the patient as inhalation air, the quantity of anaesthetic gases consumed may be radically reduced. This is a form of a so called low-flow system. The purification of the exhaled air is normally performed in a carbon dioxide absorber. The exhaled air is passed through a container containing a carbon dioxide-absorbing compound, normally anaesthetic lime, and is thereby purified from carbon dioxide.

Conventionally used absorber containers contain lime, which normally is sufficient in order to absorb carbon dioxide from the exhaled air of a patient for seven hours. Since, nowadays, most operations last a considerably shorter time, the total carbon dioxide absorption capacity is not utilized during a single operation. The quantity of carbon dioxide-absorbing substance in the container cannot, however, be reduced since an optimum effect is obtained if the absorbing substance takes up a volume which, on the whole, corresponds to one breath of the patient. During certain conditions, it is therefore very common that, for cost reduction purposes, one and the same container is used during several consequent operations.

It may also occur that the absorber container has to be temporarily disconnected during an operation in order to raise the carbon dioxide level of the patient. Thereby, an admixture of a gas which is not purified from carbon dioxide normally occurs by mechanically disconnecting the absorber container by lifting it away so that the connection lines are short-circuited for a certain time. Each such measure means a good deal of coupling work while the operation is in progress.

Each connection and disconnection of the absorber container while anaesthesia is in progress results in gas leakage if the valve manifold to which the absorber container is attachable is not provided with an automatic switch which enables continued ventilation of the patient even when the absorber is removed. Such a mechanism presumes that the affluent and the effluent from the absorber is positioned in the same end of the absorber pot. In order to minimize the resistance to the respiration gases, large cross-sectional areas of the lime container are required and for optimum utilization of the absorber compound it is important that the container provides a good distribution of the gas flow, which most easily is obtained by a cylindrical lime container. Thus, the optimum solution of the three problems: 1. Low resistance to the respiration gases, 2. No leakage when disconnecting the absorber, and 3. Maximum absorber compound efficiency, implies that a cylindrical pot with a coaxial connection to the valve manifold is used.

When a previously used absorber container is remounted on a low-flow system, it is important that the exhaled air passes through the container in the same direction as in its previous use. This is necessary in order to enable the carbon dioxide absorption to occur in an effective way in a temporarily removed container.

In contemporary anaesthetic apparatuses, all connections for inhalation and exhalation hoses are made with connections of the same type. The absorber containers are also of a type in which inlets and outlets are identical and the containers may easily be connected for a reverse direction of flow. In order to couple these connections correctly, marking of hoses, connections, inlets and outlets etc. or accurate sketches for how the systems should be composed are necessary. This risk of a mix-up also increases with the number of reconnections which have to be done when the absorber container is temporarily disconnected. In order to be completely sure that connections are done in a correct way, all connections should be verified an additional time after the completion. In operation situations, the time factor is very often deciding the outcome of the operation. Therefore, time which is consumed for the mechanical handling of equipment must be reduced as much as possible. It is therefore common that hospital staff in a stressed situation perform e.g. coupling work on the breathing systems and that the security checks during such conditions are not always performed in a completely correct manner. The risk of a mix-up of the direction of flow through the absorber container is evident in such stressed situations.

From U.S. Pat. No. 4,502,876 an anaesthetic device is prior known having a container, in which the absorber compound is enclosed by a cover in the shape of a hollow cylinder, having holes in the cylinder mantle surfaces. This results in that the cover is penetrated by the gas in substantially a radial direction. As the container has an axially directed input and output of the gas, there is a risk that the gas will not be absorbed uniformly.

### SUMMARY OF THE INVENTION

An object of the invention is to combine the advantages of the multiple-use container (low costs) and the disposable container (safe for the working environment and easy to handle) by factory-packing the correct quantity of absorber compound into a gas permeable and transparent bag (enables observation of the colour change). By choosing a degradable material for the bag, neither this nor its contents pose any environmental problem after the clinical use. The problems with the working environment in the form of dust, which as a rule are associated with the absorber compound, are minimized since the absorber compound is packed in a bag.

This object is achieved by an absorber container as is specified in the claims.

A further object of the present invention is to provide an absorber container which, by means of a rapid and simple manipulation by hand, may be connected to and disconnected from a breathing system without any risk of mixing up the direction of flow of the exhaled air through the absorber container. Furthermore, the container is designed in such a way that an exchange of absorber container or an addition of fresh absorber compound may be performed in a simple and flexible way.

Consequently, the invention comprises a carbon dioxide absorber compound packed in a gas permeable and transparent bag which may be placed in a multiple-use absorber compound container intended for the purpose, which container is an integral part of a circle system for administering anaesthesia. The multiple-use container should be designed in such a way that an exchange of a “portion bag” is easy to perform while administering anaesthesia, alternatively each circle system is provided with several multiple-use containers which may be pre-loaded with bags so that an exchange may be executed, with the multiple-use container, while anaesthesia is in progress, in the same way as currently may be done with the disposable container.

### DESCRIPTION OF THE DRAWINGS

In the following, a number of embodiments of the invention are described, whereby reference is made to the appended drawings in which:

FIG. 1 schematically shows the principle of a breathing system according to the circle principle;

FIG. 2 shows a container for absorber compound in section;

FIG. 3 shows a device according to the invention for the handling of absorber compound; and

FIG. 4 shows a device, according to a second embodiment, in the form of a vertical section through an absorber container which is an integral part of the device.

### PREFERRED EMBODIMENT

In the following, the device according to the invention is accomplished by means of specifying a number of measures as follows.

The absorber compound is prepared. It primarily consists of lime, which constitutes its active part. The lime is suitably added together with an indicator dye, a dyestuff, which changes colour at a certain proportion of absorbed carbon dioxide in the compound. Furthermore, additives for regulating the consistency of the compound may be present.

A cover for a certain quantity of compound is prepared. The cover should consist of a gas permeable material. A gauze made of an easily degradable material is preferred, preferably cotton or cellulose fibres. Alternatively, a gas permeable paper may be used.

The cover is filled with the intended quantity of compound and is sealed in order to form a bag or a sack which retains the compound so that it has a connection to the environment solely via the apertures and pores of the cover.

Anaesthetic apparatus according to the circle system is readied with a container for absorber compound which is dimensioned in order to accommodate the absorber compound, enclosed by the cover, in such a way that anaesthetic gas which has been brought into the container is forced to pass through the absorber compound inside the cover and depart from the container after the carbon dioxide uptake in order to be circulated in the system.

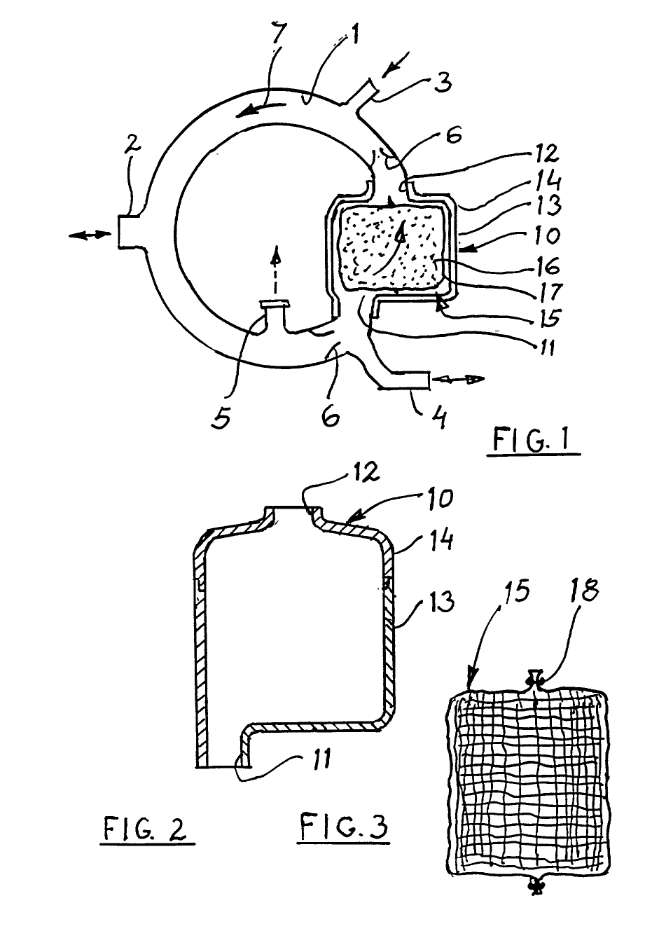

In FIG. 1 a circle system is schematically shown. In the drawing, reference numeral 1 denotes a circulation duct having a connection 2 to the patient who is to obtain anaesthesia, an inlet 3 for anaesthetic gas, “fresh gas”, a connection 4 to a system for providing a rhythmical pressure if the respiration of the patient has to be supported; which device may be a breathing bladder or a so called ventilator. Furthermore, there is a pressure relief valve 5 for the emission of surplus gas from the duct 1 as well as one-way valves 6 for directing the flow in the system in the direction of the arrow 7.

Upstream of the connection 3 for the supply of anaesthetic gas, a container 10 is inserted with an inlet opening 11 from the circulation duct 1 and an outlet opening 12 to the circulation duct. The container is divided into the actual container 13 and a lid 14. The lid 14 may be removed by being released from its connection to the circulation duct 1 or by means of the circulation duct being connected by a flexible hose.

Inside the container 10, said package 15 for absorber compound 16 is provided, being enclosed in said gas permeable cover 17.

In FIG. 2, the container 10 is shown in greater detail in a central section. The previously used reference numerals for the components of the container are also specified in FIG. 2.

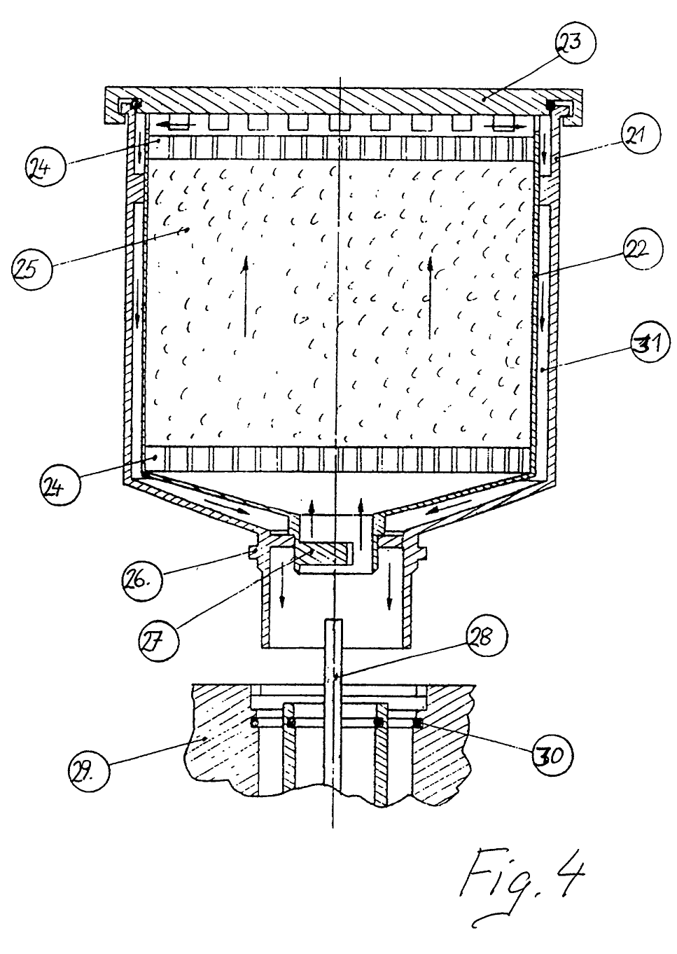

In FIG. 3, the package for absorber compound is shown in a central section. Also here, the previous reference numerals have been employed. Furthermore, it is shown that the cover, when in a state for delivery, may have a hose shape by being sealed at both ends by means of a clamp 18. As is evident from the drawing, the cover 17 is designed in a bag-like shape from a flexible and easily degradable material. By making the cover out of a homogenous material, the entire cover becomes gas permeable.

According to the method, the package 15, which may easily be handled without spreading out any absorber compound, may be inserted into the container 10 after the lid 14 has been lifted. After putting on the lid and possible reconnection to the circulation duct, the refill for the absorption of carbon dioxide acts by means of the gas streaming through the apertures and the pores of the cover and further out through the outlet 12. When the absorber compound has been consumed or when it otherwise is to be exchanged, the lid 14 is removed and the refill 15 is taken out and discarded. It may be returned to nature by being composted, whereby the lime constitutes a soil improvement agent after the cover material has decomposed.

When continued use of the system is desired, a new package 15 is inserted into the container.

In FIG. 4, an absorber container according to the invention is schematically shown, which comprises an outer container 21, an inner container 22 and a lid 23. The inner container 22 encompasses a volume filled with e.g. a granular lime material 25, which effects the absorption of carbon dioxide. The granular lime material 25 is bordered upwards and downwards by two filters 24, which together with the inner container wall delimits the absorbing volume and prevents the granular material from following the air to the inhalation or the exhalation conduits. The outer container 1 has supporting distance members on its inside, so that an air gap 31 will be formed between the containers when the inner container is placed inside the outer container. This air gap has a connection to the interior of the inner container by means of air holes in the upper portion of the inner container. When the lid 23 is screwed or is mounted in another way to the outer container, the inner container will be fixed in the correct position by means of the supporting distance members.

In the lower rim of both containers, there are connections. In the present embodiment, these connections are coaxially arranged so that the coupling portion of the inner container is completely enclosed by the coupling portion of the outer container. In this embodiment, the coupling portion of the outer container consists of a bayonet socket. Other designs, such as screw couplings or the like, are of course also conceivable.

Both couplings fit into the corresponding couplings of a valve manifold 29, in which the coupling seal is constituted of an oaring or similar sealing device, suitable for use in medical equipment. A shoulder 27 is arranged on the coupling portion of the inner container. When fastened to the valve manifold, this shoulder will press down a pin 28, which in turn is connected to and closes a bypass valve (not shown). This bypass valve automatically short-circuits the breathing system circuit when the absorber container is removed.

Before mounting the absorber container, the bypass valve in the valve manifold 29 is open and allows the exhaled air to pass over directly to the return conduit for inhalation air. Simultaneously, the couplings up to the site of the absorber container are closed. When mounting the container, the pin 28 is pressed down by the shoulder 27 and the bypass valve in the valve manifold 29 closes simultaneously as the flow to the couplings is opened. This simplifies the handling since no additional transfers have to be made when removing or inserting absorber containers and the valve manifold is also easy to use when the lime container is not applied.

The double coaxial coupling in the valve manifold 29 fits into the coupling portions of the containers, and because of the coaxial positioning, no mix-up of the different ducts can occur. The mounting procedure also comprises one single coupling operation which also makes the procedure faster. As indicated by means of arrows in FIG. 4, the exhalation air from the patient may be conducted from the inner pipe in the connection of the valve manifold 29 to the connection of the inner container 22, further through the volume in which the carbon dioxide-absorbing granular lime material 25 is situated, out through the holes in the upper end of the inner container, and back into the air gap 31 down to the outer portion of the double coupling. The purified exhalation air will thereby become available in the outer portion of the coaxial conduit in the valve manifold 29. Since the absorber container can only be mounted in a single, defined way, the inlets and the outlets can never be mixed-up. It is also implicit that the direction of flow through the container may run in the opposite direction, with the outlet via the air gap 31 and the outlet through the inner container 22. The only important thing is to ensure that the direction of flow is standardized in all equipment in which the containers might be used.

When the carbon dioxide-absorbing granular lime material 25 has been consumed, it must be exchanged before the container may be used again. The flexible construction conveys a number of possibilities. The first alternative implies that the entire container is intended to be either an expendable supply or a device which should be refilled, whereby the entire container is discarded or returned for refilling. The second alternative is that the inner container 2 is regarded as an expendable supply or device which is to be refilled at the factory, whereby the lid 23 is screwed off and the inner container 22 is discarded or returned to the factory in order to be refilled. The third alternative is that the hospital staff themselves replace the active substance in the absorber container, i.e. screw off the lid 23, take out the inner container 22, empty the consumed granular material 25, refill with fresh granular material after necessary cleaning, and place the inner container back into position. These alternatives offer great flexibility and the hospital staff may themselves choose the procedure which is best suited to their wishes.

In the above-mentioned embodiment according to FIG. 4, it is described that the granular absorber material is located in the inner container. It is of course also possible that the granular material is placed in the air gap between the inner and the outer container and that the inner container is emptied in order to function as a gas inlet or a gas outlet. The easiest way to realize this is to reduce the dimensions of the inner container into a pipe, so that the volume of the air gap between the containers becomes sufficiently large in order to be able to enclose a sufficiently large quantity of granular material. Another variant on the same theme is to let both the inner container and the air gap contain a carbon dioxide absorbing-substance. These variants are, however, not as flexible when it comes to the exchange of consumed carbon dioxide-absorbing substance, but should nevertheless be regarded as being comprised by claims 1 and 2. The invention is not limited to the embodiments described above and to the embodiments shown in the drawings, but may be varied within the scope of the subsequent claims. It is, for instance, conceivable that the disposable package according to FIG. 3 may be used in an absorber container according to FIG. 4.

1. Device for handling a carbon dioxide-absorbing compound in anaesthetic systems of the circle system type, arranged with an openable container (10) adapted for gas passage in such a way that gas, mixed with carbon dioxide from the patient who is to be connected to the system, flows through the container in order to be returned to the patient, whereby a quantity of a carbon dioxide-absorbing compound (16) is enclosed in a gas cover (17) furnished with apertures and forms a unit (15) dimensioned to be inserted into a container (10) in the anaesthetic system so that gas mixed with carbon dioxide flows through the unit when the system is used, characterized in that the entire cover (17) is made from a gas permeable and easily degradable material that the entire cover (17) has a bag-like shape.

2. Device according to claim 1, characterized in that the cover (17) is made from cellulose fibre.

3. Device according to claim 1, characterized in that the cover (17) is made from cotton gauze.

4. Device according to claim 1, characterized in that the cover (17) is made from paper.

5. Device for a circular breathing system of low-flow type for use in anaesthesia of patients and comprising an absorber container, which exhibits an outer container (21) and an inner container (22), of which one contains a carbon dioxide-absorbing compound (25), and which are mounted with the inner container inside the outer container with an air gap (31) between said containers, whereby the outer and the inner containers exhibit coupling portions which are arranged coaxially and united in a single coupling, characterized in that the inner container (2) is mounted within the outer container (1) with an air passage (11) between said containers, and that the air passage communicates with the inner of the inner container (2) by means of air holes in the part of the inner container facing away from the coupling.

6. Device according to claim 5, characterized in that when the absorber container is mounted on a valve manifold (29), a shoulder (27) influences a pin (28) in the valve manifold (29), which pin shuts a bypass valve in the valve manifold.

7. Device according to either claim 1 or claim 2, characterized in that the inner container (22) is easily dismountable for exchange of consumed carbon dioxide-absorbing compound (25).

8. Device according to claim 5, characterized in that the connection of the absorber container to the breathing system is achieved via a bayonet socket, screw joint or a similar simple coupling device.

9. Device for a circular breathing system of low-flow type for use in anaesthesia of patients and comprising an absorber container which exhibits an outer container (21) and an inner container (22), of which one contains a unit in the form of a cover (17), furnished with gas apertures, enclosing a carbon dioxide-absorbing compound and which are mounted with the inner container inside the outer container with an air gap (21) between the containers, whereby the outer and the inner containers exhibit coupling portions which are coaxially arranged and united in one single coupling,
characterized in that the absorber container as a whole has a coaxial structure and is arranged to let the air from and to the coupling portions pass through the carbon dioxide-absorbing compound in an axial direction, and that the entire cover (17) is designed to be gas permeable and made from a flexible and easily degradable material.
